(19) United States
(12) Patent Application Publication
(10) Pub. No.: US 2025/0361559 A1
(43) Pub. Date: Nov. 27, 2025

(54) UNIVERSAL PRECLINICAL BIO-DISTRIBUTION KIT FOR NK CELL THERAPY PRODUCTS

(71) Applicants: SHANGHAI MEDICILON INC., Pudong New Area, Shanghai (CN); MEDICILON (CHUANSHA) INC., Pudong New Area, Shanghai (CN)

(72) Inventors: Dengji ZHANG, Shanghai (CN); Yuqiong YUAN, Shanghai (CN); Zhenzhen BAI, Shanghai (CN); Ranran LIANG, Shanghai (CN); Chunlin CHEN, Shanghai (CN); Huanzhang ZHU, Shanghai (CN)

(21) Appl. No.: 18/861,724

(22) PCT Filed: Mar. 13, 2023
(86) PCT No.: PCT/CN2023/080965

§ 371 Date: Aug. 14, 2025

(30) Foreign Application Priority Data
May 9, 2022 (CN) 202210497094.2

(51) Int. Cl. C12Q 1/6881; C12Q 1/6809; C12Q 1/6851; C12Q 1/686; C12Q 1/6888

(52) U.S. Cl. CPC C12Q 1/6881; C12Q 1/6809; C12Q 1/6851; C12Q 1/686; C12Q 1/6888; C12Q 2600/166

(57) ABSTRACT

Provided is a universal preclinical bio-distribution detection kit for NK cell therapy products. The present invention further relates to specific use of a gene sequence set forth in SEQ ID No. 1 in distinguishing a human NK cell gene from a non-human animal gene. By means of providing a specific DNA sequence, a DNA sequence derived from the human NK cell can be specifically distinguished from a DNA sequence derived from the non-human animal gene, and based on the specific DNA sequence, a qPCR system for distinguishing the human NK cell gene from the non-human animal gene is constructed, providing a primer pair and a probe set for NK cells and derivative cell therapy products thereof. In addition, a universal preclinical bio-distribution detection kit for the NK cell therapy products is successfully designed, thereby providing convenience for the preclinical research on the NK cells and derivative cell therapy products thereof.

### TECHNICAL FIELD

The present invention relates to the fields of bio-distribution analysis, pharmacokinetics, toxicokinetics, safety evaluation of cell therapeutic products, and preclinical research, and particularly relates to a universal preclinical bio-distribution detection kit for NK cell therapeutic products.

### BACKGROUND

Natural Killer cells (NK cells) are derived from lymphoid stem cells of bone marrow, differentiation and development thereof rely on the micro-environment of bone marrow and thymus, and NK cells are mainly distributed in the bone marrow, peripheral blood, liver, spleen, lungs, and lymph nodes. Unlike T cells and B cells, NK cells are a type of lymphocyte that can non-specifically kill tumor cells and virus-infected cells without prior sensitization. In recent years, with the continuous development of cell gene therapy, T cells have been developed by researchers in the field of biomedicine into Chimeric Antigen Receptor T-Cell Immunotherapy (CAR-T) therapeutic products, and therapeutic effect of NK cells has also attracted great attention. Currently, a variety of NK cell-transformed products have been used in pharmaceutical research and development, such as CAR-NK products based on virus vector transduced cells, CAR-NK cell therapy products based on extracellular coupling, dual-target or multi-target CAR-NK products, and gene-edited NK cell products, etc.

NK products for cell therapy are inseparable from preclinical pharmacokinetics and bio-distribution studies. CARs expressed on NK cells due to introduction (transduction) of viral vectors can be specifically detected based on the sequence of the transferred gene, i.e., the sequence of the CAR portion. The current commonly used technical approach is to detect specific CARs using qPCR technology for each novel CAR-NK product based on the CAR sequence. However, such detection method based on transferred genes requires the construction of independent qPCR detection methods for different molecules for each transferred gene, resulting in a huge workload for early preclinical research and a long time consumption, which has unfavorable time costs for rapidly advancing early research. Therefore, establishing a universal method system that can specifically and directly distinguish genomic DNA from human NK cell and genomic DNA from experimental animals such as mice, not only provides fast and convenient preclinical research, but also has important industrial value and economic benefits.

Furthermore, CAR-NK products directly coupled with CAR portions outside the cell, or other transformed NK cell therapeutic products, are not suitable for a method of constructing qPCR based on CAR sequences. For example, for CAR products that are directly coupled, since CAR itself is already expressed as a protein product for coupling, DNA level detection methods based solely on CAR are no longer applicable. In order to characterize the pharmacokinetics and bio-distribution behavior of NK cells themselves, it is necessary to construct a quantitative PCR method based on the gene sequence of NK cells themselves that can specifically distinguish human NK cell genes from animal genes, based on intrinsic NK cell gene sequences. How to find a specific DNA sequence for the above distinction is a common challenge encountered in this field. Specifically, although CD56 molecule is a marker molecule on the surface of NK cells, the gene of this molecule is actually present in mouse and human NK cells, as well as other cells or tissues. How to find a gene-specific sequence and apply to the pharmacokinetics and distribution of NK cells, and successfully construct a corresponding detection method has always been an important challenge faced by those skilled in the art.

### SUMMARY OF THE INVENTION

In view of the aforementioned issues, the present invention innovatively identifies and proposes a specific DNA sequence that can specially distinguish DNA sequences derived from human NK cells from those derived from non-human animal genes. Based on this specific DNA sequence, the present invention constructs a qPCR system to distinguish human NK cell genes from non-human animal genes, provides a primer pair and a probe sets for NK cell and derivative cell therapy products, and successfully design a universal preclinical bio-distribution detection kit for NK cell therapy products, thereby facilitating preclinical research on NK cell and derivative cell therapy products. Specifically, the following technical problems need to be addressed in the present invention:
1. Specificity. Particularly, how to find a gene sequence that is differentially expressed with animals such as mice in the genes of thousands of human NK cells. There are thousands of genes in both NK cell genomes and animal genomes. Finding differentially expressed gene not only requires extensive comparative analysis, but also rigorous experimental validation to determine whether the differentially expressed gene can distinguish sequences with differences. qPCR technology is merely a technical method or technical carrier. qPCR technology can be fully applied and have practical value only when combined with a specific gene sequence. Identifying a specific sequence and designing a qPCR method based on the specific sequence is essential for achieving pharmacokinetic and bio-distribution analysis of NK cells.
2. Methodology design. Based on the qPCR quantitative detection technology for DNA, methodological design is carried out for personalized and specific differentially expressed gene sequences, such as primer design, probe design, and target sequence selection.
3. Configuration of reaction system. For example, the control and optimization of components and proportions in the reaction system.
4. Application of critical buffers in the reaction system. Compared with basic scientific research, the present invention is anchored in industrial application, whereas alterations in components may impact the usability of the kit. Therefore, the reaction system of the present invention does not require direct water treatment or dilution of samples as in basic scientific research to meet the requirements, but requires a precise and rigorous analysis system, emphasizing changes in various details, thus ensuring that the method and kit meet practical experimental verification.
5. Determination of reaction conditions. The reaction conditions include the temperature and time of pre-denaturation, denaturation, annealing, and elongation (extension).
6. Optimization of various conditions, preferably, to improve the sensitivity and amplification efficiency of the methodology. For example, to overcome the interference of matrix components in template (i.e., genomic DNA) extraction solutions from different individuals and matrix sources.

In a first aspect, the present invention provides a specific use of a gene sequence set forth in SEQ ID No. 1 in distinguishing human NK cell genes from non-human animal genes.

In a second aspect, the present invention provides a use of a gene sequence set forth in SEQ ID No. 1 in the preparation of a universal preclinical bio-distribution detection kit for NK cell therapy products. In particular, the present invention provides a use of a reagent for detecting a gene sequence set forth in SEQ ID No. 1 in the preparation of a universal preclinical bio-distribution detection kit for human NK cell therapy products. In particular, the present invention provides a use of a reagent for detecting a gene sequence set forth in SEQ ID No. 1 in the preparation of a universal preclinical bio-distribution detection kit for human NK cell therapy products.

In a third aspect, the present invention provides a primer pair for specifically distinguishing human NK cell genes from non-human animal genes. The primer pair is used to amplify a target gene sequence set forth in SEQ ID No. 1; the primer pair consists of a Primer-F forward primer and a Primer-R reverse primer; the Primer-F forward primer is a primer set forth in SEQ ID No. 2, or is a DNA molecule that is subjected to substitution and/or deletion and/or addition of one or several nucleotides and has the same function as SEQ ID No. 2; the Primer-R reverse primer is a primer set forth in SEQ ID No. 3, or is a DNA molecule that is subjected to substitution and/or deletion and/or addition of one or several nucleotides and has the same function as SEQ ID No. 3.

Preferably, the forward primer has a melting temperature of 55° C.±1° C.; and the reverse primer has a melting temperature of 55° C.±1° C.

Preferably, a difference in melting temperature between the forward primer and the reverse primer is not higher than 2° C. If the difference in melting temperature between the forward and reverse primers is too large, it may cause asynchronous annealing.

In a fourth aspect, the present invention provides use of a primer pair for specifically distinguishing human NK cell genes from non-human animal genes in the preparation of a universal preclinical bio-distribution detection kit for NK cell therapy products.

In a fifth aspect, the present invention provides a primer probe set for specifically distinguishing human NK cell genes from non-human animal genes. The primer probe set comprises a TaqMan probe where a fluorescent group is coupled at the 5′ end and a quencher group is coupled at the 3′ end of a gene sequence set forth in SEQ ID No. 4, and a primer pair described above.

Preferably, the probe has a melting temperature of 65° C.±1° C.

Preferably, the melting temperature of the probe is 10° C.±1° C. higher than that of the Primer-F forward primer or the Primer-R reverse primer.

In a sixth aspect, the present invention provides use of a primer probe set mentioned above for specifically distinguishing human NK cell genes from non-human animal genes in the preparation of a universal preclinical bio-distribution detection kit for NK cell therapy products.

In a seventh aspect, the present invention provides a universal preclinical bio-distribution detection kit for NK cell therapy products. The kit comprises a primer pair mentioned above for specifically distinguishing human NK cell genes from non-human animal genes. Alternatively, the present invention provides a universal preclinical bio-distribution detection kit for NK cell therapy products, comprising a primer probe set mentioned above for specifically distinguishing human NK cell genes from non-human animal genes.

Preferably, the universal preclinical bio-distribution detection kit for NK cell therapy products further comprising a standard plasmid.

Preferably, a standard curve is constructed using a standard plasmid, a qPCR detection system is constructed using a primer pair or a primer probe set, a specific target gene sequence set forth in SEQ ID No. 1 is obtained through an amplification reaction, and a difference between human NK cell genes and non-human animal genes is identified.

Preferably, the amplification reaction conditions are: 94° C. to 96° C./3 minutes to 4 minutes; 94° C. −96° C./15 seconds to 20 seconds; 54° C. to 56° C./0.75 minute to 1 minute; cycle times: 35 times to 40 times. In some technical solutions, the amplification reaction conditions are: 95° C./3 minutes; 95° C./15 seconds; 55° C./1 minute; cycle times: 40 times.

### SEQUENCE LISTING

The gene sequences for the human KLRC1 gene segment and primers and probe therefor described herein are listed in the sequence listing entitled “RevisedSequenceListing.xml,” created on Aug. 5, 2025, and having a file size of 11 KB, the entirety of which is hereby incorporated by reference for all purposes.

### BRIEF DESCRIPTION OF DRAWINGS

FIG. 1 is an electrophoresis photo of the homo2 gene after amplification; wherein, M is a molecular marker (DNA marker).

FIG. 2 is a schematic diagram of the agarose gel electrophoresis identification of PCR amplification products; wherein, M is a molecular marker (DNA marker), N is a blank control, and X1, X2, and X3 respectively are the genomic DNA of three blank mice, and R is the genomic DNA of human NK cells.

FIG. 3 is an amplification curve diagram of the kit of the present invention.

FIG. 4 is a standard curve diagram of the kit of the present invention.

### DETAILED DESCRIPTION OF EMBODIMENTS

Unless otherwise specified, all experimental methods disclosed in the present invention adopt conventional techniques in the field of molecular biology, biochemistry, analytical chemistry, and related fields.

Unless otherwise defined, all technical and scientific terms used herein have the same meanings as those familiar to a person skilled in the art. In addition, any methods and materials similar or equivalent to those described herein can be applied to the present invention. The preferred implementation methods and materials described herein are only examples and are not limited to them.

Select a target DNA sequence for detection. The present invention proposes to use a specific sequence set forth in SEQ ID No. 1 (from a segment of KLRC1 gene) as the target DNA sequence for detection. KLRC1 is a type II trans-membrane protein (43kD) that is mainly expressed on the membrane of Natural Killer cells (NK) cells and belongs to the NK receptor family (NKG2 family). The protein encoded by this gene belongs to the killer cell lectin-like receptor family, and is a group of trans-membrane proteins that are preferentially expressed on NK cells. The inventors attempted a variety of gene sequences during the research and development stage, such as CD56, homo2, B-actin, etc. However, although these sequences can be amplified on NK cells, but can also be amplified to corresponding sequences in the mouse genome. In the prior art, KLRC1 as an asthma biomarker has been amplified by qPCR to explore the correlation between KLRC1 and asthma. However, the present invention proposes for the first time that the KLRC1 gene sequence set forth in SEQ ID No. 1 is used to distinguish gene sequences of human NK cells from gene sequences of other animals species. Other species include but are not limited to mice, cynomolgus monkeys, rats, New Zealand rabbits, and beagles.

Based on the KLRC1 gene sequence shown in SEQ ID No. 1, a primer pair and primer probe set have been designed to specifically distinguish human NK cell genes from animal genes. The design principles of the primer pair and primer probe set are to select and verify the reliability of combination of the primer pair and probe based on melting temperature (Tm value), GC content (the ratio of guanine and cytosine among the four bases of DNA), size of upstream primer end sequence and primer sequence, as well as characteristics of primer terminal (end) base and probe terminal (end) base.

The primer pair is used to amplify the target detection gene sequence shown in SEQ ID No. 1. The primer pair consists of a Primer-F forward primer and a Primer-R reverse primer. The Primer-F forward primer is a primer shown in SEQ ID No. 2, or is a DNA molecule that is subjected to substitution and/or deletion and/or addition of one or several nucleotides and has the same functionality as SEQ ID No. 2; the Primer-R reverse primer is a primer shown in SEQ ID No. 3, or s a DNA molecule that is subjected to substitution and/or deletion and/or addition of one or several nucleotides and has the same functionality as SEQ ID No. 3. In some technical solutions, the Tm value of the primer pair is controlled at around 55° C. As an example, the Tm value of the reverse primer is designed to be 54.75° C., and the Tm value of the forward primer is designed to be 55.81° C.

The primer probe set consists of a primer pair and a probe. Specifically, the probe is a TaqMan probe where a fluorescent moiety is coupled at the 5′ end of the gene sequence shown in SEQ ID No. 4 and a quencher group is coupled at the 3′ end the gene sequence shown in SEQ ID No. 4. In some embodiments, a fluorescent group is 6-FAM (6-Carboxyfluorescein), and a quencher group is TAMRA (Carboxytetramethylrhodamine). The present invention adopts the TaqMan probe method to design a probe molecule, which is a TaqMan probe coupled with a luminescent group 6-carboxyfluorescein (6-FAM) at the 5′ end and a quenching group carboxytetramethylrhodamine (TAMRA) at the 3′ end of a single stranded DNA. A free intact probe has no fluorescence signals. The fluorescence emitted by the luminescent group is absorbed and quenched by the quenching group. When the probe is hydrolyzed, the luminescent and quenching groups move away, allowing for the detection of fluorescence signals. In some technical solutions, the Tm value of the probe is about 65° C., such as 64.34° C.

Preferably, the Tm value of the Taqman probe is 10° C. higher than that of the primer pair. This difference in Tm values ensures that the probe and primers bind sequentially to the template chain, thereby ensuring correct probe shearing.

Next, an exemplary description will be given to illustrate the universal preclinical bio-distribution detection kit for NK cell therapy products according to the present invention. This kit uses a qPCR method to amplify and detect the target DNA in standard samples, quality control samples and/or test samples. At the beginning of the reaction, the template chain forms a single chain through thermal denaturation. The TaqMan probe is annealed first with the template chain, and the primer is subsequently annealed onto the template. Afterwards, the chain is extended. During the extension process, Taq enzyme exhibits activity of 5′-3′ exonuclease, and cleaves the probe base by base from the 5′ end when encountering a probe, thus separating the luminescent group from the quenching group. Therefore, the fluorescence detection system can receive fluorescence signals. Every time a DNA strand is amplified, a fluorescent molecule is formed. The accumulation of fluorescence signals and the formation of PCR products are synchronous.

The universal preclinical bio-distribution detection kit for NK cell therapy products includes a primer pair and a Taqman probe based on the KLRC1 gene in NK cells. As an example, the concentration of the primer pair is 10 mol/L and the concentration of the probe is 10 mol/L. Optionally, the kit also contains a positive control. The positive control is a nucleic acid sample containing KLRC1 gene expression. Of course, the kit may also contain negative controls. The negative control is a nucleic acid sample without KLRC1 gene expression.

Optionally, the kit also comprises a DNA diluent.

Optionally, the kit also comprises a standard plasmid.

Optionally, the kit also comprises a pre-mixed solution. The pre-mixed solutions can be prepared by self-configuring or purchased commercially. As an example, the premix solution may be qPCR Taqman Probe Master Mix.

During the usage of the kit, user can supplement genomic DNA from the corresponding matrix source according to the type of test sample. For example, if the sample is mouse whole blood, genomic DNA extracted from mouse whole blood prepared with DNA diluent can be used to prepare standard samples and QC (Quality Control) samples. During detection, water can be added to supplement the volume of the reaction system, and the extracted DNA template can be added to the sample for analysis and detection, which is time-saving and convenient.

In some technical solutions, the usage method of the kit comprises the following steps: (1) Add sample. Add genomic cDNA sample, positive control sample or negative control sample into a PCR tube containing a PCR reaction system to obtain a corresponding sample reaction tube, positive reaction tube or negative reaction tube. The PCR reaction system contains the aforementioned KLRC1 gene detection primer. (2) PCR reaction. The reaction tube is placed on a PCR instrument, and reaction condition parameters such as temperature, time, and cycle times are set to perform the PCR reaction. (3) After the PCR reaction is completed, analyze the results.

In the development of this kit, the present invention design a method for real-time quantitative amplification of a portion of the KLRC1 gene sequence based on the Taqman qPCR technique, which meets the specificity requirements of distinguishing human NK cell genomes from animals genomes, such as mice. Based on this method, a reaction system and reaction conditions are established, and the qPCR kit is optimized, validated, and constructed, in order to be applicable for early preclinical pharmacokinetic and bio-distribution studies of NK cells in mice and even other animal species.

When an embodiment gives a numerical range, it should be understood that, unless otherwise specified in the present invention, both endpoints of each numerical range and any value between the endpoints can be selected. Unless otherwise defined, all technical and scientific terms used in the present invention shall have the same meanings as those commonly understood by person skill in the art. In addition to the specific methods, equipment, and materials used in the embodiments, any existing methods, equipment, and materials that are similar or equivalent to the methods, equipment, and materials described in the embodiments of the present invention may also be used to implement the present invention based on the knowledge of those skilled in the art and the description of the present invention. The following illustrates the process of exploring and improving the present invention through nodal embodiments.

### Instruments and Equipment

1) ABI fluorescence quantitative PCR instrument (7500 or equivalent alternative version);
2) Fluorescence quantitative PCR 8-strip tube & cap (BBI, F602004-0001, or equivalent substitute);
3) Centrifuge (Sangon Biotech (Shanghai) Co., Ltd., Super Mini Dancer, or equivalent substitute);
4) 96-well PCR plate (AXYGEN, PCR-96-AB-C, or equivalent alternative).

### Reagents

1) Standard plasmid pUC-GW-Kan-NK (Suzhou GeneWiz Biotechnology Co., Ltd., clone ID: ZA5917-1/A751715, Lot #A751715-20211228 or other batches, ≤−10° C.);
2) qPCR Taqman probe Master mix (Yeasen Biotechnology (Shanghai) Co., Ltd., Cat #: 11205ES08, Lot #H8001170, ≤−10° C.);
3) DNA diluent (Cat #: B639270, Sangon Biotech (Shanghai) Co., Ltd.).

### Example 1

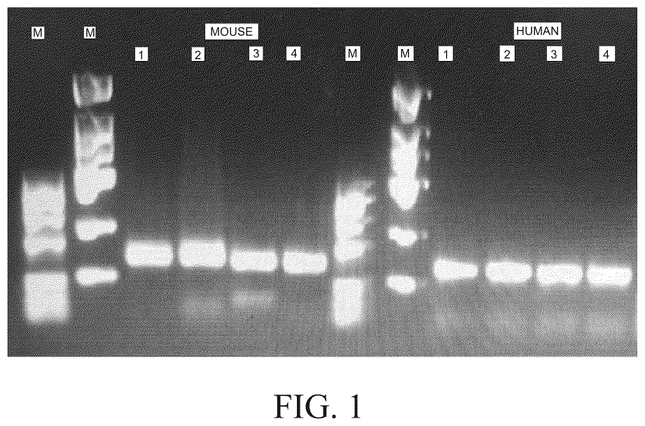

The surface marker molecules of NK cells, such as CD16 and CD56, are present in both human and non-human species such as mice and have little difference. Therefore, using these genes cannot distinguish human NK cells from non-human species animals, nor can they be used as marker gene sequences for constructing NK cell pharmacokinetic distribution. The inventor also used homo2 gene to distinguish human NK cell genes from non-human animal genes, but the homo2 gene is not only amplified specifically in human NK cells, but can also be significantly amplified in the mouse genome. The electrophoresis photo after amplification is shown in FIG. 1. Wherein, M is Marker; lanes 1, 2, 3, and 4 on the left are the amplification results of genomic DNA from blood sources of four different mice; lanes 1, 2, 3, and 4 on the right are the amplification results of genomic DNA sequences of four human NK cells. It can be seen that the homo2 primer sequence cannot specifically amplify only in human genomic DNA.

### Example 2

Prior to determining the target sequence for amplification in the present invention, the inventor attempted different sequence fragments of KLRC1 and designed a plurality of primer pairs, and also used gene sequences from the same family as KLRC1, such as the KLRK1 gene sequence or other genes such as IL-15 and 3-actin sequences, and designed a plurality of primers pairs based on these gene sequences to determine the specificity and applicability of the selected amplification sequence. This example uses KLRC1-1, KLRC1-2, KLRC1-3, KLRC1-4, KLRK1-1, KLRK1-2, KLRK1-3, KLRK1-4, IL-15, and 3-actin primer pairs to amplify the corresponding genes sequence, and screen the target sequence and primer pairs based on the amplification effect. The reaction system and reaction conditions refer to the implementation steps and conditions of the method described in the present invention, and the only difference in the reaction system is SYBR™ Green dye instead of the probe. Because the target sequence and primer pair have not yet been determined, using SYBR™ Green dye can preliminarily determine the specificity of the primer pair for amplification based on the dissolution curve. Specifically, the following primer pairs are selected, and both human NK cell genomic DNA and mouse genomic DNA are used as templates. The specificity of amplification is determined based on the dissolution curve of qPCR amplification through SYBR™ Green dye method. Primer pairs with no peak or no single main peaks in the dissolution curve cannot be selected, and the corresponding amplified target sequence cannot be selected. If there are a plurality of main peaks or no main peaks in the dissolution curve, it indicates that the gene sequence is not suitable for the present invention. If a single main peak appears in the dissolution curve, it is considered that the gene sequence can be used as a candidate gene sequence for constructing a specific Taqman qPCR method. Genomic DNA (gDNA) was randomly extracted from two mice (designated as Mouse 1 and Mouse 2) and human NK cells for amplification. The threshold cycle number (CT) and dissolution curve results are shown in Table 1.

TABLE 1

| Primer Name | Sample | CT Value | Dissolution Curve |
|---|---|---|---|
| KLRC1-1 | Genomic DNA of Mouse 1 | No CT Value | No Peaks |
| | Genomic DNA of Mouse 2 | No CT Value | No Peaks |
| | Genomic DNA of Human NK cells | 21 | There is one Main Peak |
| KLRC1-2 | Genomic DNA of Mouse 1 | No CT Value | No Peaks |
| | Genomic DNA of Mouse 2 | 34 | There is one Main Peak |
| | Genomic DNA of Human NK Cells | 21 | There is one Main Peak |
| KLRC1-3 | Genomic DNA of Mouse 1 | No CT Value | No Peaks |
| | Genomic DNA of Mouse 2 | No CT Value | No Peaks |
| | Genomic DNA of Human NK Cells | 23 | There is one Main Peak |
| KLRC1-4 | Genomic DNA of Mouse 1 | No CT Value | No Peaks |
| | Genomic DNA of Mouse 2 | No CT Value | No Peaks |
| | Genomic DNA of Human NK Cells | 20 | There are two Main Peaks and |
| | | | Non-specific Amplification |
| KLRK1-1 | Genomic DNA of Mouse 1 | No CT Value | No Peaks |
| | Genomic DNA of Mouse 2 | No CT Value | No Peaks |
| | Genomic DNA of Human NK Cells | No CT Value | There is one Main Peak |
| KLRK1-2 | Genomic DNA of Mouse 1 | No CT Value | There is one Main Peak |
| | Genomic DNA of Mouse 2 | No CT Value | There is one Main Peak |
| | Genomic DNA of Human NK Cells | No CT Value | There is one Main Peak |
| KLRK1-3 | Genomic DNA of Mouse 1 | No CT Value | No Peaks |
| | Genomic DNA of Mouse 2 | No CT Value | No Peaks |
| | Genomic DNA of Human NK Cells | 21 | There is one Main Peak |
| KLRK1-4 | Genomic DNA of Mouse 1 | No CT Value | No Peaks |
| | Genomic DNA of Mouse 2 | 32 | There is one Main Peak |
| | Genomic DNA of Human NK Cells | 21 | There is one Main Peak |
| IL-15 | Genomic DNA of Mouse 1 | No CT Value | No Peaks |
| | Genomic DNA of Mouse 2 | No CT Value | No Peaks |
| | Genomic DNA of Human NK Cells | No CT Value | No Peaks |
| β-actin | Genomic DNA of Mouse 1 | 25 | There is one Main Peak |
| | Genomic DNA of Mouse 2 | 25 | There is one Main Peak |
| | Genomic DNA of Human NK Cells | 18 | There is one Main Peak |

It can be seen that when amplifying β-actin, there are peaks (amplification) in both mouse and human genomic DNA, and this is a housekeeping gene that has absolutely no specificity for humans and mice, so it cannot be applied; IL-15 is not amplified in both human NIK cells and mouse genomic DNA, and also cannot be applied; The dissolution curves of KLRC1, KLRC3, KLRK1, and KLRK3 all have a main peak, indicating preliminary specificity and potential application value in distinguishing human NK cells from mouse.

### Example 3

The four primer pairs corresponding to the four gene sequences of KLRC1, KLRC3, KLRK1, and KLRK3 selected in Example 2 have the potential to be used to distinguish human NK cell genes from non-human animal genes, that is, they have specific amplification on human NIK cells but no specific amplification in mouse genomic DNA. In this example, the amplified product is further sequenced using a sequencer The sequencing results are shown in Table 2.

TABLE 2

| | Sequencing | | |
|---|---|---|---|
| Primer Name | Results | Compare Results of Homologous Genes | Similarity |
| KLRC1-1F | Overlapping | Homo sapiens natural killer group protein 2-A (NKG2-A) gene, | 100% |
| | Peaks | complete coding sequences | |
| KLRC1-1R | Normal | Select seq AB073871.2 | 100% |
| | | Homo sapiens KLRCI gene for NKG2-A, partial coding | |
| | | sequence (select gene bank sequence number AB073871.2, i.e., | |
| | | member I of Human killer cell lectin-like receptor subfamily C, | |
| | | or natural killer cell activating receptor 2A, partial coding | |
| | | sequence) | |
| KLRC1-3F | Normal | Homo sapiens KLRCI gene for NKG2-A, partial coding | 100% |
| | | sequence (human killer cell lectin-like receptor subfamily C, or | |
| | | member I of natural killer cell activating receptor 2A, partial | |
| | | coding sequence) | |
| KLRC1-3R | Overlapping | Select seq AH003673.2 | 98.86% |
| | Peaks | Homo sapiens chromosome 12 C-lectin type II protein (NKG2- | |
| | | A) gene, complete coding sequence (selected from gene bank | |
| | | sequence number AH003673.2, human killer cell lectin-like | |
| | | receptor subfamily C, member I of natural killer cell activating | |
| | | receptor 2A, complete coding sequence) | |
| KLRK1-1F | Ploy | No Matching Gene | No Data |
| KLRK1-1R | Ploy | Select seq NG_027762.1 | 89.92% |
| | | Homo sapiens killer cell lectin like receptor K1 (KLRK1), | |
| | | RefSeqGene on chromosome 12((Select gene bank sequence | |
| | | number NG_027762.1, that is, human killer cell lectin-like | |
| | | receptor subfamily K, member I, referring to gene sequence | |
| | | located on chromosome 12) | |
| KLRK1-3F | Overlapping | Homo sapiens killer cell lectin-like receptor K1 (KLRK1), | 94.45% |
| | Peaks | RefSeqGene on chromosome 12 (human killer cell lectin-like | |
| | | receptor subfamily K, member I, referring to gene sequence | |
| | | Located on chromosome 12) | |
| KLRK1-3R | Sequencing | Unable to Compare | No Data |
| | Failed | | |

The gene sequence of KLRC1-1F is shown in SEQ ID No. 2. The gene sequence of KLRC1-1R is shown in SEQ TD No. 3. The gene sequence of KLRC1-3F is shown in SEQ ID No. 5. The gene sequence of KLRC1-3R is shown in SEQ ID No. 6. The gene sequence of KLRK1-1F is shown in SEQ ID No. 7. The gene sequence of KLRK1-1R is shown in SEQ ID No. 8. The gene sequence of KLRK1-3F is shown in SEQ ID No. 9. The gene sequence of KLRK1-3R is shown in SEQ ID No. 10.

Table 2 demonstrates that the primer pairs of KLRK1 (member I of human killer cell lectin-like receptor subfamily K) cannot accurately align with the genes on its homologous human NK cells, and therefore is not applicable. Among the two sets of primers pairs designed based on KLRC1 (Member I of Killer Cell Lectin-Like Receptor Subfamily C, also known as NKG2A), the first primer pair (KLRC1-1F; KLRC1-1R) shows better performance, could be sequenced well, and is able to find the homologous sequences of human NK cells with 100% similarity in the gene library. This indicates that the forward and reverse primers (KLRC1-1F and KLRC1-1R) designed for KLRC1 in the present invention are optimal sequences for amplifying NK cells.

### Example 4

On the basis of determining the amplified target sequence in Example 3, this example further designs and screens a specific probe sequence to design a specific Taqman qPCR quantitative method. Based on the implementation steps and conditions of the method described in the present invention, the same target sequence (i.e., template) containing different copy number gradients was added to the reaction system for amplification (template loading amount was luL), with the difference being the design of Tagman probes with different sequences. The sequence of KLRC1-probe 1 is shown in SEQ ID No. 11. The sequence of KLRC1-probe 2 is shown in SEQ ID No. 4, and a fluorescent group is coupled at 5′ end and a quenching group is coupled at 3′ end of the sequence. The probe amplification results are shown in Table 3.

TABLE 3

| | Target | Cycle | | Target | Cycle |
|---|---|---|---|---|---|
| | Sequence | Threshold | | Sequence | Threshold |
| | Copy | Value | | Copy | Value (CT |
| | Number | (CT Value) | | Number | Value) |
| KLRC1-Probe | 10⁸ | 25 | KLRC1-Probe | 10⁸ | 22 |
| 1 | 10⁸ | 26 | 2 | 10⁸ | 21 |
| | 10⁸ | 26 | | 10⁸ | 21 |
| | 10⁷ | 29 | | 10⁷ | 26 |
| | 10⁷ | 31 | | 10⁷ | 26 |
| | 10⁷ | 31 | | 10⁷ | 26 |
| | 10⁶ | 34 | | 10⁶ | 30 |
| | 10⁶ | 34 | | 10⁶ | 30 |
| | 10⁶ | 34 | | 10⁶ | 30 |
| | 10⁵ | 37 | | 10⁵ | 33 |
| | 10⁵ | 38 | | 10⁵ | 33 |
| | 10⁵ | 39 | | 10⁵ | 33 |
| | 10⁴ | Non-Detect | | 10⁴ | 38 |
| | 10⁴ | Non-Detect | | 10⁴ | 38 |
| | 10⁴ | Non-Detect | | 10⁴ | 38 |

Under the same reaction conditions, it is ultimately determined through parameter comparison that the probe claimed by the present invention (KLRC1-Probe2) has the best design effect. When the copy number of the target sequence is on the order of 108 magnitude, the Ct value of the probe sequence claimed by the present invention is lower than that of the comparative probe; when the copy number of the target sequence is on the order of 105 magnitude, the Ct value of the comparative probe is close to the total number of cycles, while the Ct value of the probe sequence claimed by the present invention is 33, which has a window compared to the total number of cycles; when the copy number of the target sequence is on the order of 104 magnitude, the comparative probe exceeds the detection limit and is undetectable, while the probe sequence claimed by the present invention has a lower detection limit, and can still be detected even when the copy number is on the order of 104 magnitude. The probe sequence protected by the present invention has greater potential and value for further optimization and use. As described above, the present invention designs a plurality of primers pairs based on KLRC1, performs amplification and sequencing, and then selects a target sequence that can be detected with high fidelity, and ultimately determines the corresponding claimed primers. After the primers are determined, a plurality of probes are designed and a probe with good sensitivity and excellent amplification efficiency is selected through comparison as an important component of the detection method and kit of the present invention.

### Example 5

### The Specificity Identification of the Primer and the Screened Gene of the Present Invention (Agarose Gel Electrophoresis Identification)

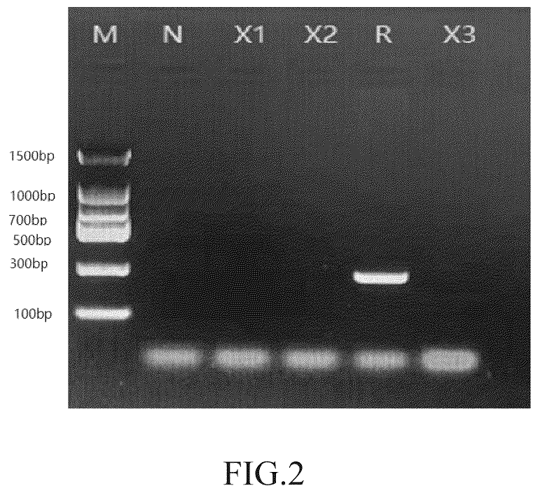

It can be seen from FIG. 2 that through the agarose gel electrophoresis identification of the primer pair and the PCR amplification product of the method of the present invention, the results show that bright bands with the size similar to the detected target DNA merely appear at the target location, indicating that the primer pair for KLRC1 provided by the method of the present invention, can amplify the specific target DNA fragment well, and can effectively distinguish the differences between human NK cells and mouse samples based on the KLRC1 sequence, with only human NK cells amplifying to the target band, while there is no amplification in mouse DNA samples. Therefore, the gene screened by the present invention can serve as a marker gene sequence for identifying the pharmacokinetic distribution of human NK cells in mice.

### Example 6

Prepare a reaction mixture (Master Mix) of the reaction system. The amount of template DNA added can vary between 1 μL to 5 μL. In actual use, the amount of each component of the reaction mixture can be adjusted appropriately and proportionally according to the actual number of test samples. In this example, the reaction mixture includes: 10 μL of qPCR Taqman Probe Master Mix, 0.4 μL of KLRC1-F, 0.4 μL of KLRC1-R, 0.2 μL of probe-KLRC1, 0.4 μL of ROX, 2 μL of template DNA, and 6.6 μL of ultrapure distilled water. The total volume of the reaction system is 20 μL. The function of ROX is to correct fluorescence fluctuations unrelated to PCR, thereby minimizing inter-well differences to the greatest extent possible. In practical applications, if the amount of template DNA added is changed, corresponding changes can be made to the volume of ultrapure distilled water. For example, if the amount of template DNA added is 1 μL, the amount of ultrapure distilled water added can be adjusted to 5.6 μL.

An amplified target DNA sequence is inserted into a standard plasmid as a standard sample to establish a standard curve. First, the standard plasmid is diluted to 1.900×10⁹ copies/μL, and then is diluted with a 10-fold series of DNA dilutions containing genomic DNA derived from blank un-administered mouse blood or tissue to form a series of standard samples with different copy number concentrations for forming the standard curve of this method. The standard curve exhibits a linear relationship based on the Ct value and the Log value of sample concentration (in copy number). Quality control samples and test samples can be calculated based on the standard curve fitted by regression with standard samples, obtaining the Log value of the unknown sample concentration via Ct value of the sample, thus the sample concentration can be determined. The preparation of a standard solution is shown in Table 4. The preparation of quality control samples is shown in Table 5.

TABLE 4

| | | Volume of | Volume of | | Target Solution | |
|---|---|---|---|---|---|---|
| Serial | | Source | Genomic DNA | Total | Serial | Final |
| Number of | Dilution | Reagent | of Mixed Blank | Volume | Number of | Concentration |
| Source Tube | Factor | (μL) | Mouse (μL) | (μL) | Target Tube | (Copy/μL) |
| Stock A | 10 | 5 | 45 | 50 | Standard 01 | 1.900 × 10⁸ |
| Standard 01 | 10 | 10 | 90 | 100 | Standard 02 | 1.900 × 10⁷ |
| Standard 02 | 10 | 10 | 90 | 100 | Standard 03 | 1.900 × 10⁶ |
| Standard 03 | 10 | 10 | 90 | 100 | Standard 04 | 1.900 × 10⁵ |
| Standard 04 | 10 | 10 | 90 | 100 | Standard 05 | 1.900 × 10⁴ |
| Standard 05 | 10 | 10 | 90 | 100 | Standard 06 | 1.900 × 10³ |
| Standard 06 | 10 | 10 | 90 | 100 | Standard 07 | 1.900 × 10² |
| Standard | NA | NA | 90 | 90 | BG | 0 |

TABLE 5

| | | Volume of | Volume of | | Target Solution | |
|---|---|---|---|---|---|---|
| Serial | | Source | Genomic DNA of | Total | Serial | Final |
| Number of | Dilution | Reagent | Mixed Blank | Volume | Number of | Concentration |
| Source Tube | Factor | (μL) | Mouse (μL) | (μL) | Target Tube | (Copy/μL) |
| Stock A | 10 | 5 | 45 | 50 | ULOQ-1 | 1.900 × 10⁸ |
| ULOQ-1 | 1.25 | 20 | 5 | 25 | HQC-1 | 1.520 × 10⁸ |
| HQC-1 | 50 | 5 | 245 | 250 | Int.A-1 | 3.040 × 10⁶ |
| Int.A-1 | 20 | 5 | 95 | 100 | MQC-1 | 1.520 × 10⁵ |
| MQC-1 | 16 | 5 | 75 | 80 | Int.B-1 | 9.500 × 10³ |
| Int.B-1 | 19.92 | 10 | 189.2 | 199.2 | LQC-1 | 4.770 × 10² |
| LQC-1 | 2.51 | 10 | 15.1 | 25.1 | LLOQ-1 | 1.900 × 10² |

The volume of the standard solutions and quality control samples prepared above can be adjusted proportionally.

The data from each analysis batch is collected using Sequence Detection Software v1.5.1 (ABI7500) or an upgraded version, and processed using SoftMax software. Linear regression is performed on the Ct values of amplification curves at each concentration point of the standard curve and the Log values of theoretical concentrations (in copies) to determine the standard curve. The concentration (in copies) of quality control samples and/or test samples can be calculated from the standard curve. If the quality control sample and/or the test sample have been diluted, the measured concentration (in copies) can be multiplied by the corresponding dilution factor to obtain the final measured concentration (in copies). The test results are shown in Table 6.

TABLE 6

| | Cycle Threshold Value |
|---|---|
| Standards | (CT Value) |
| Standard 01 | 13 |
| Standard 02 | 17 |
| Standard 03 | 21 |
| Standard 04 | 24 |
| Standard 05 | 28 |
| Standard 06 | 31 |
| Standard 07 | 35 |

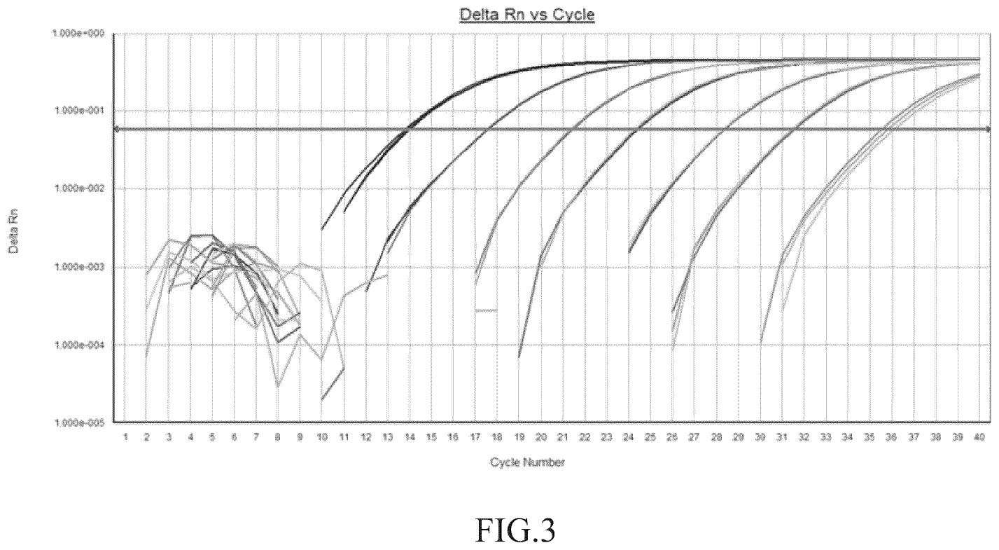

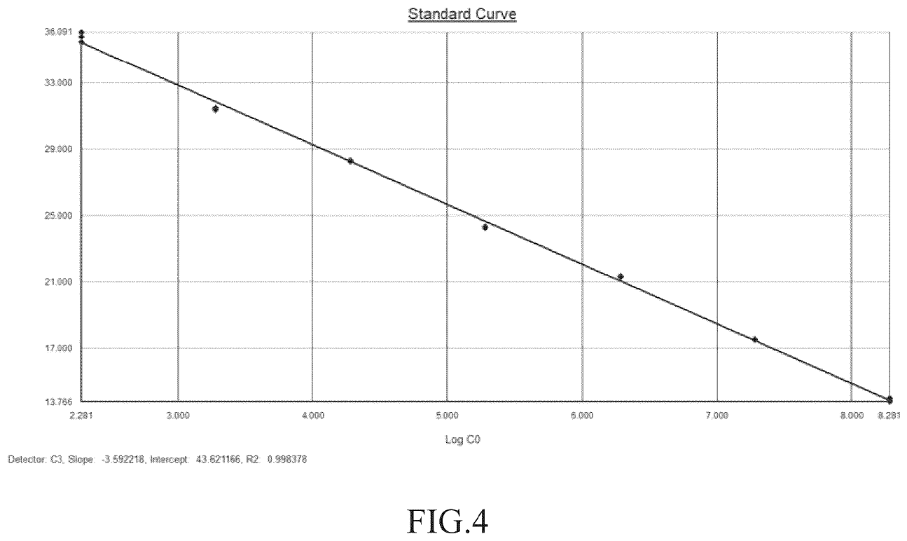

After a series of condition optimizations and comparisons, the technical solution of the present invention is determined and examined. The results show that the amplification curve exhibits a good performance when conducting experiments based on the standard curve setting conditions, technical system, and reaction conditions of the present invention. It can be seen from FIG. 3 and FIG. 4 that the R² value of the standard curve is 0.998, close to 1. The difference in Ct values between different concentration gradients can be widened and evenly distributed, and the slope is −3.59, close to −3.32, based on this, the amplification efficiency reaches 90%. Based on this, other implementation effects of the present invention also are investigated.

### Example 7

### Assessment of the Implementation Effects in Terms of Accuracy and Precision

Accuracy and precision testing was conducted based on the technical solution of the present invention. A standard curve was configured and three independent sets of quality control samples (QC) were prepared. The results showed that the standard curve (STD) has excellent accuracy and precision in fitting, and the recovery rate of the quality control samples is consistent with the test results of the same level of quality control samples in different sets of three sets of quality control samples. The accuracy and precision of the standard curve are shown in Table 7. The accuracy and precision of the quality control samples are shown in Tables 8 and 9.

TABLE 7

| Sample of | Concentration | | | | | Average | CV of Copy | |
|---|---|---|---|---|---|---|---|---|
| Standard | of Copy | | Average CT | CV of CT | | Copy | Number | Accuracy |
| Curve | Number | CT Value | Value | Value (%) | Copy Number | Number | (%) | (%) |
| Standard | 190,000,000 | 14.906 | 14.943 | 0.479 | 164896418.7 | 160968013.2 | 4.73 | 84.7 |
| Sample 1 | | 14.898 | | | 165815686.9 | | | |
| | | 15.026 | | | 152191934.1 | | | |
| Standard | 19,000,000 | 18.088 | 18.046 | 0.208 | 19566382.2 | 20130200.57 | 2.499 | 105.9 |
| Sample 2 | | 18.016 | | | 20533105.81 | | | |
| | | 18.034 | | | 20291113.69 | | | |
| Standard | 1,900,000 | 21.309 | 21.406 | 0.404 | 2262614.464 | 2122105.722 | 5.862 | 111.7 |
| Sample 3 | | 21.474 | | | 2026015.312 | | | |
| | | 21.436 | | | 2077687.392 | | | |
| Standard | 190,000 | 25.034 | 24.928 | 0.649 | 186656.715 | 201184.145 | 11.152 | 105.9 |
| Sample 4 | | 24.742 | | | 227023.544 | | | |
| | | 25.009 | | | 189872.175 | | | |
| Standard | 19,000 | 28.172 | 28.336 | 0.546 | 22816.996 | 20512.174 | 10.463 | 108 |
| Sample 5 | | 28.479 | | | 18571.147 | | | |
| | | 28.358 | | | 20148.379 | | | |
| Standard | 1,900 | 31.703 | 31.93 | 0.631 | 2143.073 | 1852.211 | 13.868 | 97.5 |
| Sample 6 | | 32.088 | | | 1656.493 | | | |
| | | 32.000 | | | 1757.066 | | | |
| Standard | 190 | 35.434 | 35.462 | 1.025 | 176.169 | 176.19 | 23.825 | 92.7 |
| Sample 7 | | 35.84 | | | 134.223 | | | |
| | | 35.114 | | | 218.177 | | | |

TABLE 8

| Quality | | | | | |
|---|---|---|---|---|---|
| Control | | QC-Set1 | | | |
| (QC) | Concentration of | | Copy Number | | |
| Samples | Copy Number | Average Copy Number | Results | CV of Copy Number (%) | Recovery Rate(%) |
| QC1 | 190,000,000 | 164308636 | 175682887 | 7.115 | 86.5 |
| | | | 152324511 | | |
| | | | 164918510 | | |
| QC2 | 152,000,000 | 143881216 | 149805108 | 3.578 | 94.7 |
| | | | 140485088 | | |
| | | | 141353452 | | |
| QC3 | 152,000 | 173875.531 | 164362.796 | 5.358 | 114.4 |
| | | | 182980.231 | | |
| | | | 174283.566 | | |
| QC4 | 477 | 594.743 | 513.429 | 14.032 | 124.7 |
| | | | 590.617 | | |
| | | | 680.182 | | |
| QC5 | 190 | 218.875 | 217.142 | 3.935 | 115.2 |
| | | | 228.222 | | |
| | | | 211.26 | | |

| Quality | | | | | |
|---|---|---|---|---|---|
| Control | | QC-Set2 | | | |
| (QC) | | | Copy Number | | |
| Samples | Concentration | Average Copy Number | Results | CV of Copy Number CV (%) | Recovery Rate(%) |
| QC1 | 190,000,000 | 173,509,096 | 172557403.1 | 5.582 | 91.3 |
| | | | 183635786.6 | | |
| | | | 164334096.8 | | |
| QC2 | 152,000,000 | 149,475,959 | 147821601 | 0.995 | 98.3 |
| | | | 149905480.9 | | |
| | | | 150700796.1 | | |
| QC3 | 152,000 | 178196.083 | 190381.563 | 6.335 | 117.2 |
| | | | 176114.181 | | |
| | | | 168092.506 | | |
| QC4 | 477 | 650.085 | 664.467 | 10.891 | 136.3 |
| | | | 712.594 | | |
| | | | 573.196 | | |
| QC5 | 190 | 239.359 | 286.935 | 19.192 | 126 |
| | | | 235.884 | | |
| | | | 195.258 | | |
| | | QC-Set3 | | | |
| | | | Copy Number | | |
| Sample | Concentration | Average Copy Number | Results | CV of Copy Number CV (%) | Recovery Rate(%) |
| QC1 | 190,000,000 | 182987476.9 | 186087392 | 8.249 | 96.3 |
| | | | 166583798 | | |
| | | | 196291241 | | |
| QC2 | 152,000,000 | 158799928.3 | 153934787 | 2.66 | 104.5 |
| | | | 160935517 | | |
| | | | 161529481 | | |
| QC3 | 152,000 | 183454.916 | 175642.967 | 14.717 | 120.7 |
| | | | 161222.584 | | |
| | | | 213499.196 | | |
| QC4 | 477 | 574.187 | 582.874 | 6.308 | 120.4 |
| | | | 605.274 | | |
| | | | 534.411 | | |
| QC5 | 190 | 238.206 | 265.059 | 30.74 | 125.4 |
| | | | 155.346 | | |
| | | | 294.214 | | |

TABLE 9

| Sample | Concentration | QC Batch-to-batch Accuracy | QC Batch-to-batch Precision |
|---|---|---|---|
| QC1 | 190,000,000 | 91.4 | 5.4 |
| QC2 | 152,000,000 | 99.2 | 5.0 |
| QC3 | 152,000 | 117.4 | 2.7 |
| QC4 | 477 | 127.1 | 6.5 |
| QC5 | 190 | 122.2 | 5.0 |

A standard curve was configured and three independent quality control samples were prepared in the experiment to examine the implementation effect. The results show that the accuracy and precision of the standard curve were good, and each individual set of the three sets of quality control samples had good accuracy and precision, and when compared between the three sets of quality control samples, the three sets of quality control samples had excellent interbatch accuracy and precision.

### Example 8

### The Implementation Effect of Overcoming Matrix Effect in the Present Invention

Genomic DNA was extracted from the livers and blood of ten different mice individuals, and a standard plasmid containing an amplified target gene fragment was added to the genomic DNA extraction solution of each mice individual at a certain copies concentration (152,000 copies). Based on the technical solution of the present invention, qPCR amplification detection was carried out, and the implementation effect was evaluated by the recovery rate of the number of copies of the target gene added to each mice genomic DNA. The test results are shown in Table 10.

TABLE 10

| | | | CV of CT | | Average | | CV of | |
|---|---|---|---|---|---|---|---|---|
| | Concentration | | value | Average | Result | Result | Copies | Recovery |
| Sample | (Copies) | CT Value | (%) | (CT) | (Copies) | (Copies) | (%) | Rate (%) |
| Ma01 | 152,000 | 22.214 | 0.151 | 22.192 | 127269.617 | 125354.3 | 2.27 | 83.7 |
| | | 22.208 | | | | 125861.8 | | |
| | | 22.153 | | | | 130592.8 | | |
| Ma02 | 152,000 | 22.298 | 0.242 | 22.237 | 123476.129 | 118460.9 | 3.594 | 81.2 |
| | | 22.196 | | | | 126891.4 | | |
| | | 22.217 | | | | 125076.1 | | |
| Ma03 | 152,000 | 21.219 | 0.351 | 21.296 | 232802.426 | 245033.8 | 5.04 | 153.2 |
| | | 21.302 | | | | 231730.6 | | |
| | | 21.368 | | | | 221642.8 | | |
| Ma04 | 152,000 | 22.231 | 0.186 | 22.207 | 125962.113 | 123960.9 | 2.798 | 82.9 |
| | | 22.231 | | | | 123894.2 | | |
| | | 22.16 | | | | 130031.2 | | |
| Ma05 | 152,000 | 22.167 | 0.142 | 22.197 | 126785.19 | 129376.2 | 2.12 | 83.4 |
| | | 22.23 | | | | 124011 | | |
| | | 22.195 | | | | 126968.3 | | |
| Ma06 | 152,000 | 22.241 | 0.154 | 22.219 | 124977.642 | 123112.5 | 2.32 | 82.2 |
| | | 22.236 | | | | 123502.7 | | |
| | | 22.179 | | | | 128317.7 | | |
| Ma07 | 152,000 | 22.23 | 0.159 | 22.201 | 126451.899 | 124019.4 | 2.387 | 83.2 |
| | | 22.212 | | | | 125506.3 | | |
| | | 22.162 | | | | 129830 | | |
| Ma08 | 152,000 | 22.172 | 0.059 | 22.177 | 128563.244 | 128941.3 | 0.885 | 84.6 |
| | | 22.166 | | | | 129463.3 | | |
| | | 22.191 | | | | 127285.1 | | |
| Ma09 | 152,000 | 21.888 | 0.303 | 21.812 | 135830.109 | 128858.4 | 4.451 | 89.4 |
| | | 21.77 | | | | 139634.4 | | |
| | | 21.777 | | | | 138997.5 | | |
| Ma10 | 152,000 | 21.751 | 0.281 | 21.724 | 144180.779 | 141485.5 | 4.213 | 94.9 |
| | | 21.654 | | | | 151136.2 | | |
| | | 21.767 | | | | 139920.6 | | |

The results showed that among the 10 randomly selected mice, the recovery rate of the copies of the target gene added to 9 extracted genomic DNA ranges from 80% to 100%, indicating that this method has no matrix effect interference and has good effect.

In summary, through homology alignment of sequence information using the NCBI gene database, the inventor found that only the human KLRC1 gene sequence has 100% similarity to the sequence screened by the claimed kit. Therefore, from the alignment result of the bio-informatic sequence alignment, it can be determined that the gene sequence set forth in SEQ ID No. 1 involved in the present invention can distinguish human NIK cell sequences from those of all non-human species, and serves excellently as a specific sequence for the universal analysis of NIK cells biological distribution. In pharmaceutical research, cynomolgus monkey is a primate sample commonly used in preclinical studies of drugs. However, through homology alignment of sequence information, it was found that the cynomolgus monkey has no homology with the gene sequence set forth in SEQ ID No. 1 obtained by the screening of the present invention. Other closely related animal species (such as Yunnan snub-nosed monkey and rhesus macaque) respectively have only 940 and 920 homology, with the gene sequence set forth in SEQ ID No. 1 utilized in the present invention. Mice that also do not have homology are economically viable in early preclinical studies (as confirmed by the data in the present invention), and mice are a commonly used animal species in preclinical studies. This demonstrates that the present invention follows the 3R principle of reducing, optimizing, and replacing the use of preclinical research animals in pharmaceutical development.

TABLE 11

| | | Similarity | |
|---|---|---|---|
| Description | species | (%) | Serial Number |
| Homo sapiens chromosome 12 clone, | Homo sapiens | 100 | AC277791.1 |
| complete sequence | | | |
| Homo sapiens chromosome 12 C-lectin type | Homo sapiens | 100 | AH003673.2 |
| II protein (NKG2-A) gene, complete code | | | |
| sequence | | | |
| Homo sapiens 12 BAC RP11-291B21 | Homo sapiens | 100 | AC068775.52 |
| (Roswell Park Cancer Institute Human BAC | | | |
| Library) complete sequence | | | |
| Homo sapiens natural killer group protein 2- | Homo sapiens | 100 | AF023840.1 |
| A (NKG2-A) gene, complete code sequence | | | |
| PREDICTED: Rhinopithecus bieti NKG2- | Rhinopithecus | 94 | XM_017866450.1 |
| A/NKG2-B type II integral membrane | bieti | | |
| protein-like (LOC108525490), mRNA | | | |
| Macaca mulatta BAC CH250-201L20 ( ) | Macaca | 92 | AC201868.4 |
| complete sequence | mulatta | | |
| Homo sapiens KLRCI gene for NKG2-A, | Homo sapiens | 100% | AB073871.2 |
| partial code sequence | | | |

The inventor uses the kit claimed in this invention, constructs a system targeting the corresponding animal species, and conducts testing applications on SD rats, New Zealand rabbits, and Beagle dogs. Genomic DNA was extracted from each of the three aforementioned animal species. A standard plasmid containing an amplified target gene fragment was added at a certain copies concentration (152,000 copies) to each genomic DNA extraction solution. Based on the technical solution of the present invention, qPCR amplification detection is carried out to investigate the recovery rate of the target gene with the added copies to each genomic DNA to evaluate the implementation effect, and to explore whether the kit has the problem of matrix interference detection. The detection data of SD rat, New Zealand rabbit, and beagle samples are as follows:

TABLE 12

| | | | CV of | | | | | |
|---|---|---|---|---|---|---|---|---|
| | | | CT | | Average | | Copies | |
| | Concentration | CT | Value | Average | Result | Result | of CV | Recovery |
| Sample | (Copies) | Value | (%) | (Copies) | (Copies) | (Copies) | (%) | Rate (%) |
| SD Rat | 152,000 | 23.002 | 0.288 | 22.931 | 179557.384 | 170858.871 | 4.539 | 118.1 |
| Sample | | 22.871 | | | | 187019.526 | | |
| | | 22.92 | | | | 180793.755 | | |
| New | 152,000 | 23.007 | 0.539 | 22.882 | 186091.099 | 170257.858 | 8.498 | 122.4 |
| Zealand | | 22.878 | | | | 186130.034 | | |
| Rabbit | | 22.76 | | | | 201885.406 | | |
| Sample | | | | | | | | |
| Beagle | 152,000 | 23.088 | 0.245 | 23.088 | 161083.998 | 160924.174 | 3.908 | 106.0 |
| Sample | | 23.144 | | | | 154869.998 | | |
| | | 23.031 | | | | 167457.822 | | |

The above data confirms that the present invention is also applicable to animal species such as SD rats, New Zealand rabbits, and Beagle dogs.

1. A method for specifically distinguishing human natural killer (NK) cell genes from non-human animal genes using a gene sequence set forth in SEQ ID No. 1.

2. (canceled)

3. A primer pair for specifically distinguishing human NK cell genes from non-human animal genes,
wherein the primer pair is used to amplify a target gene sequence set forth in SEQ ID No. 1;
the primer pair consists of a Primer-F forward primer and a Primer-R reverse primer;
the Primer-F forward primer is a primer set forth in SEQ ID No. 2, or is a DNA molecule that is subjected to substitution and/or deletion and/or addition of one or several nucleotides and has the same function as SEQ ID No. 2; and
the Primer-R reverse primer is a primer set forth in SEQ ID No. 3, or is a DNA molecule that is subjected to substitution and/or deletion and/or addition of one or several nucleotides and has the same function as SEQ ID No. 3.

4. The primer pair according to claim 3, wherein
the Primer-F forward primer has a melting temperature of 55° C.±1° C.; and the Primer-R reverse primer has a melting temperature of 55° C.±1° C., and
a difference in melting temperature between the Primer-F forward primer and the Primer-R reverse primer is not higher than 2° C.

5-6. (canceled)

7. A primer probe set for specifically distinguishing human NK cell genes from non-human animal genes, wherein the primer probe set comprises a Tagman probe where a luminescent group is coupled at the 5′ end and a quencher group is coupled at the 3′ end of a gene sequence set forth in SEQ ID No. 4, and a primer pair for specifically distinguishing human NK cell genes from non-human animal genes according to claim 3.

8. The primer probe set according to claim 7, wherein
the probe has a melting temperature of 65° C.±1° C., and
the melting temperature of the probe is 10° C.±1° C. higher than that of the Primer-F forward primer or the Primer-R reverse primer.

9-10. (canceled)

11. A universal preclinical bio-distribution detection kit for NK cell therapeutic products, comprising a primer pair for specifically distinguishing human NK cell genes from non-human animal genes according to claim 3.

12. A universal preclinical bio-distribution detection kit for NK cell therapy products, comprising a primer probe set for specifically distinguishing human NK cell genes from non-human animal genes according to claim 7.

13. The universal preclinical bio-distribution detection kit for NK cell therapy products according to claim 12, further comprising a standard plasmid.

14. The universal preclinical bio-distribution detection kit for NK cell therapy products according to claim 13, wherein a standard curve is constructed using a standard plasmid, a qPCR detecting system is constructed using a primer pair or a primer probe set, a specific target gene sequence set forth in SEQ ID No. 1 is obtained through amplification reaction, and a difference between human NK cell genes and non-human animal genes is identified.

15. The universal preclinical bio-distribution detection kit for NK cell therapy products according to claim 14, wherein the amplification reaction conditions are:
94° C. to 96° C./3 minutes to 4 minutes;
94° C. to 96° C./15 seconds to 20 seconds;
54° C. to 56° C./0.75 minute to 1 minute; and
cycle times: 35 times to 40 times.


SEQUENCE LISTING (11 sequences)

SEQ ID NO: 1 - DNA, 217 bp, Homo sapiens
  source 1..217: mol_type = genomic DNA
gtgatgtcagggactgtactctataataacagtagttaagaagttaataaatggtttatatacaggattccctagtgcaattaaaagggtgaggtggagaacgagtattggagctcattttgcattaaataatctaaagctctttctctgattttcttcaagcctaaacacgtttcttatgaatatttgttttaaaaaaagatttttaggccgggcg

SEQ ID NO: 2 - DNA, 18 bp, synthetic construct
  source 1..18: mol_type = other DNA
cgcccggcctaaaaatct

SEQ ID NO: 3 - DNA, 21 bp, synthetic construct
  source 1..21: mol_type = other DNA
gtgatgtcagggactgtactc

SEQ ID NO: 4 - DNA, 34 bp, synthetic construct
  source 1..34: mol_type = other DNA
aggattccctagtgcaattaaaagggtgaggtgg

SEQ ID NO: 5 - DNA, 18 bp, synthetic construct
  source 1..18: mol_type = other DNA
cgcccggcctaaaaatct

SEQ ID NO: 6 - DNA, 20 bp, synthetic construct
  source 1..20: mol_type = other DNA
gtgatgtcagggactgtact

SEQ ID NO: 7 - DNA, 23 bp, synthetic construct
  source 1..23: mol_type = other DNA
ttggtatccttatacgccatgag

SEQ ID NO: 8 - DNA, 24 bp, synthetic construct
  source 1..24: mol_type = other DNA
catgccaactaaaaataaacaaaa

SEQ ID NO: 9 - DNA, 23 bp, synthetic construct
  source 1..23: mol_type = other DNA
ttgtatatggtgtgagatgaggg

SEQ ID NO: 10 - DNA, 28 bp, synthetic construct
  source 1..28: mol_type = other DNA
caggaataaatccatgtattatagccaa

SEQ ID NO: 11 - DNA, 34 bp, synthetic construct
  source 1..34: mol_type = other DNA
cacttgaaatcacccactaaatgaggtaggctag